(19) United States
(12) Patent Application Publication
(10) Pub. No.: US 2003/0216649 A1
(43) Pub. Date: Nov. 20, 2003

(54) Audible output sonogram analyzer

(76) Inventor: Mark Nowicki, Tobermory (CA)

Correspondence Address: ANISSIMOFF & ASSOCIATES, RICHMOND NORTH OFFICE CENTRE, SUITE 201, 235 NORTH CENTRE RD., LONDON, ON, N5X 4E7, CA

(21) Appl. No.: 10/382,494

(22) Filed: Mar. 7, 2003

Related U.S. Application Data
(60) Provisional application No. 60/362,069, filed on Mar. 7, 2002.

(51) Int. Cl. A61B 8/00

(52) U.S. Cl. 600/443

(57) ABSTRACT

A method and apparatus for creating digital representations of sounds from printed sonograms. The method and apparatus are especially useful for audibly reproducing bird songs from printed sonograms in guidebooks to aid in the identification of birds. The apparatus is preferably conveniently sized to fit in one hand, is portable, and is capable of being used outdoors. An optical image acquisition element is used to acquire a digitized image of the printed sonogram which is then analyzed to produce a digital representation of the sound that may be stored for selective playback as an audible output and used, for example, as an aid in the identification of birds. The optical image acquisition element may be mounted within the housing or may be mounted within a separate housing. The method is also adapted for use with a pocket computer or personal digital assistant (PDA). Data logging and data transfer features are also included.

### FIELD OF THE INVENTION

The invention relates to the audible reproduction of sounds depicted by printed sonograms. In particular, the invention relates to an apparatus and method for translating printed sonograms into digital representations of sounds for the purpose of, for example, aiding in the identification of birds.

### BACKGROUND OF THE INVENTION

In the field of ornithology, guidebooks are typically used to identify birds encountered outdoors. The guidebooks usually contain written descriptions of the bird's plumage with color pictures. Birds are not always readily visible, and a bird's call is often used to audibly aid in identification of a particular bird. The call may be described in the guidebook using words and is often visually depicted in the form of a printed sonogram. A significant amount of experience is required to mentally translate the visual depictions of the bird call into an audible form. Not all amateur ornithologists possess the required experience; accordingly, it is desirable to audibly reproduce the bird calls for readily comparing them to those heard outdoors, thereby aiding in bird identification.

Sound reproduction is normally accomplished by re-playing recordings stored on, for example, magnetic media such as cassette tapes. These recordings may be replayed on a portable device at a desired volume level. To build a library of recordings takes a significant amount of time and effort, and although an extensive library exists in printed form as sonograms, none is currently readily available on magnetic media. Also, since there may be many species of birds within a given habitat, it is not always possible to carry all desired recordings. With magnetic media, a time consuming search is required to identify the desired bird call, which is further complicated by not having a picture of the bird readily available for correlation with the recording. Since ornithologists normally carry a guidebook containing all required identification information in any event, it is desirable to audibly reproduce the bird calls from the printed sonograms in order to avoid the unnecessary encumbrance and complication of supplementary recordings.

A printed sonogram is a visual depiction of a sound on a frequency versus time plot. The frequency of a sound is analogous to its pitch. The amplitude, or loudness, of the sound is represented on the sonogram by the intensity or darkness of the image. For example, a thin vertical line of constant intensity represents a short impulsive sound with equal loudness at a multitude of frequencies. A thin horizontal line of constant intensity represents a long tonal sound of continuous loudness. In practice, a sonogram is “smeared”, in time and in frequency, mainly due to the nature of the equipment that produced the sonogram. The thin vertical line therefore becomes a widened vertical stroke of intensity fading in time. As well, the thin horizontal line becomes a widened horizontal stroke with fading edges.

The present invention seeks to address the aforementioned disadvantages of recorded bird calls and the challenges of mentally interpreting printed sonograms by providing an apparatus and method for converting printed sonograms into audible sounds.

### SUMMARY OF THE INVENTION

According to the present invention, there is provided a method comprising: providing a printed sonogram corresponding to a sound, the printed sonogram comprising visible strokes corresponding to the frequency and amplitude of sounds occurring at a given time; optically acquiring a digitized image of the sonogram; and, processing the digitized image to convert the digitized image to a digital representation of the sound. The digitized image may comprise a series of side by side columns, each column having a plurality of vertically stacked pixels, each pixel having an intensity and may be processed as each column of pixels is optically acquired from the digitized image. Alternatively, the digitized image may be optically acquired in its entirety and stored in a memory buffer to allow greater flexibility in processing the digitized image. The digitized image is processed to determine frequency, amplitude and time data for each stroke based on the intensity of the pixels and the data are assembled into a digital representation of the sound depicted by the sonogram. The digital representation of the sound may be formatted for use with a personal computer. The digital representation of the sound may be stored in a storage location and a plurality of digital representations of sounds may be stored. The digital representations may be transmitted to a data receiving device. An audible signal may be generated from the digital representation of the sound to thereby audibly reproduce the sound depicted by the sonogram.

According to another aspect of the invention there is provided an apparatus comprising: a housing, the housing sized to fit in one hand; an electrical power source mounted within the housing for providing electrical power to the apparatus; an optical image acquisition element for optically acquiring a digitized image of a printed sonogram corresponding to a sound desired to be reproduced, the printed sonogram comprising visible strokes corresponding to the frequency and amplitude of sounds occurring at a given time; and, a microprocessor mounted within the housing, the microprocessor for processing the digitized image and programmed to convert the digitized image to a digital representation of the sound.

According to yet another embodiment of the invention, there is provided a method comprising: making a preliminary identification of a bird based on hearing a first sound produced by the bird; selecting a printed sonogram based on the preliminary identification of the bird from a book having a plurality of sonograms cross referenced with bird identities, the printed sonogram comprising visible strokes corresponding to the frequency and amplitude of sounds occurring at a given time, the printed sonogram corresponding to a second sound; optically acquiring a digitized image of the sonogram; storing the digitized image in a memory buffer; processing the digitized image to convert the digitized image to a digital representation of the second sound; generating an audible signal from the digital representation of the second sound to thereby audibly reproduce the second sound; and, comparing the second sound with the first sound to confirm the preliminary identification of the bird.

According to yet another embodiment of the invention, the optical image acquisition element comprises: a downwardly oriented uniform array of photodetectors positioned above a portion of the printed sonogram and having a length and width; a light source arranged to evenly illuminate the portion of the printed sonogram under the array; a lens positioned between the array and the portion of the printed sonogram, the lens for focussing an optical image of the printed sonogram onto the array; and, a rotational element in contact with the printed sonogram for providing an indication of displacement of the array along the sonogram. Preferably, the photodetectors in the array are positioned immediately adjacent one another and are closely spaced. The array of photodetectors may be a contact image sensor. The light source may comprise an array of light sources, such as a photodiode array. The lens may be flat or cylindrical in shape. There may be more than one lens. One of the lenses may be an anamorphic lens. The lens may be positioned a distance from the array. The distance between the lens and the printed sonogram may be variable. The rotational element may be a roller or a wheel.

In one embodiment, the digitized image may be processed by: determining a numerical intensity value for each pixel in a first column of the series; determining the presence of a columnar section of a stroke on the digitized image based on the numerical intensity value of the pixels in the column and recording time data corresponding to the lateral position of the column on the digitized image; determining the midpoint of the columnar section of the stroke and recording frequency data corresponding to the height of the midpoint on the digitized image; summing the numerical intensity values of each pixel in the columnar section of the stroke and recording amplitude data corresponding to the summed numerical intensity values; forming a numerical list of data for the column containing the recorded frequency, time and amplitude data; and, repeating the previous steps for each of the columns in the series and assembling the numerical list of data for each column into a digital representation of the sound.

The optical image acquisition element may include a video input device, a line scanner, a charge coupled device (CCD), or a photodetector array, such as a contact image sensor. The optical image acquisition element may be mounted in the housing or may be separate from the housing and removably connected therewith. The optical image acquisition element is used for acquiring a digitized image of a printed sonogram for processing. The digitized image may be stored in a memory buffer, such as a Random Access Memory (RAM) memory buffer or “flash” EEPROM.

A microprocessor is programmed to process the stored digitized image and obtain values for time, amplitude, and frequency from the sonogram based on scanning image columns sequentially along the horizontal axis. A list of numerical data corresponding to the values is created for each column. The lists of numerical data for each column of the digitized image are assembled into a digital representation of the sound depicted by the sonogram. The digital representation of the sound may be stored in a storage location. The storage location may be on a memory element located either inside or outside the housing. A plurality of digital representations of sounds may be stored.

A digital representation of the sound may be selected from a storage location for conversion to an audible output using an audible signal generator. The audible signal generator generally comprises a digital to analog converter, an amplifier, and an audio output transducer. In one embodiment, the audible signal generator also comprises an audio generator and a variable attenuator. Some or all of these components may be mounted within the housing; for example, the audio output transducer may be a speaker located within the housing or may alternatively be mounted within an ear piece to allow unobtrusive reproduction of bird sounds without disturbing any birds that may be present.

In one embodiment, a pocket computer or personal digital assistant (PDA) may be used. The pocket computer or PDA may, for example, be used to provide the housing, power source, memory element, and microprocessor. An optical image acquisition element may be removably connected with the pocket computer or PDA. The audible signal generator or a portion thereof may be provided with the pocket computer or PDA. By using the pocket computer or PDA, information about a bird sighting may be logged, for example, type of bird sighted, time and location of the bird sighting, GPS position data, atmospheric conditions, etc. The pocket computer or PDA can also be used to provide access to a library of information, such as digitized images of sonograms or digital representations of sounds. The pocket computer or PDA also allows sharing of data with other pieces of electronic equipment using either direct connections, such as a cable or infrared link, or indirect connections, such as the Internet.

Further features of the invention will be described or will become apparent in the course of the following detailed description.

### BRIEF DESCRIPTION OF THE DRAWINGS

In order that the invention may be more clearly understood, preferred embodiments thereof will now be described by way of example with reference to the accompanying drawings, in which:

FIG. 1 shows an example of a printed sonogram;

FIG. 2 shows an embodiment of an apparatus according to the present invention;

FIG. 3 shows a photograph of the inside of the housing of an embodiment of an apparatus according to the present invention;

FIG. 4 shows an embodiment of an apparatus according to the present invention having a variable focal length;

FIG. 5a shows a square grid;

FIG. 5b shows a square grid as viewed through a Bravais anamorphic lens;

### DETAILED DESCRIPTION OF PREFERRED EMBODIMENTS

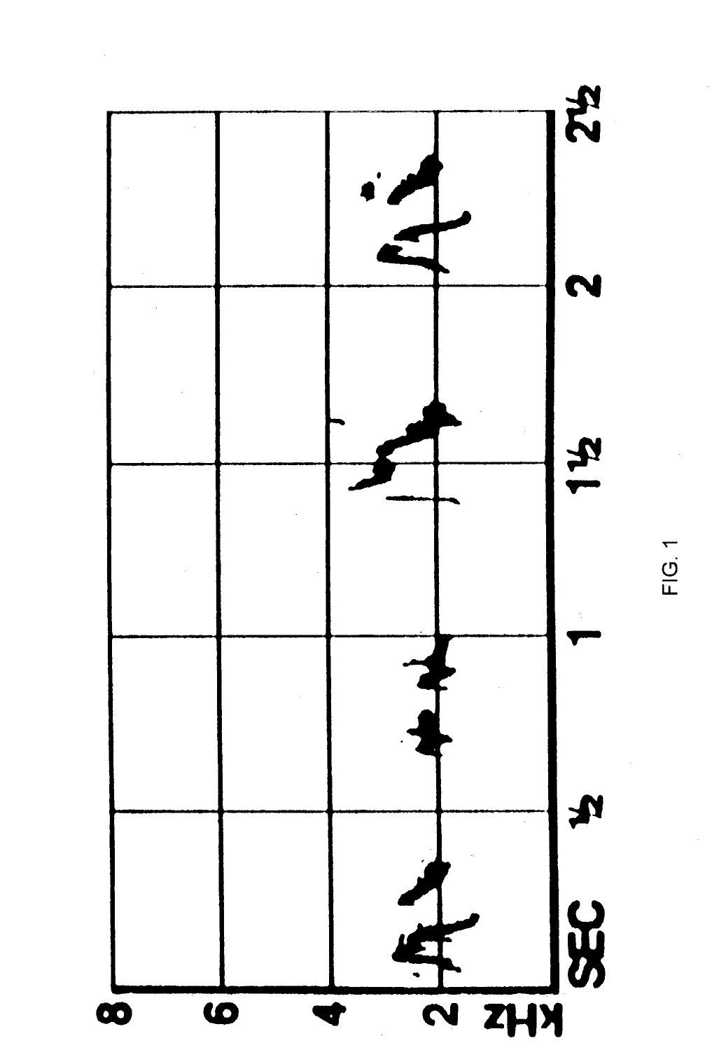

The strokes on a sonogram are visual depictions of the energy of a sound distributed in frequency and time. An example of a printed sonogram depicting a sound made by a bird is shown in FIG. 1 (Golden®/A Guide to Field Identification Birds of North America. Golden Press, New York, 1983, p.14, which is herein incorporated by reference). The ordinate axis corresponds to frequency, or pitch, and the abscissa corresponds to time. The intensity, or darkness, of the strokes on the sonogram corresponds to the amplitude, or loudness, of the sounds. A columnar slice of the sonogram conceptually represents an amplitude versus frequency profile of a sound at a particular instant in time. By examining the sonogram as a succession of thin columnar slices, frequency, amplitude, and time data can be extracted for each column of the sonogram.

In one embodiment, the digitized image is processed by: determining a numerical intensity value for each pixel in a first column of the series; determining the presence of a columnar section of a stroke on the digitized image based on the numerical intensity value of the pixels in the column and recording time data corresponding to the lateral position of the column on the digitized image; determining the midpoint of the columnar section of the stroke and recording frequency data corresponding to the height of the midpoint on the digitized image; summing the numerical intensity values of each pixel in the columnar section of the stroke and recording amplitude data corresponding to the summed numerical intensity values; forming a numerical list of data for the column containing the recorded frequency, time and amplitude data; and, repeating the previous steps for each of the columns in the series and assembling the numerical list of data for each column into a digital representation of the sound.

The foregoing description refers to the reconstruction of a single sinusoid of varying frequency and varying amplitude from a sonogram with a single stroke. Many audio signals, however, are more complex and include multiple frequency components that are usually, but not necessarily, harmonically related. These frequency components are visible in the sonogram as stacked strokes. In order to reproduce the nuances, or tonal character, of a particular sound, all frequency components present in the sonogram must be reconstructed and audibly emitted. The previously described method is equally applicable to these types of complex audio signals and may be applied to simultaneously generate a number of frequency components from a sonogram with stacked strokes, storing each of them as a list of numerical data as previously described. The resulting numerical data is then combined into a digital representation of a sound that may be amplified and audibly emitted as described above, thereby reproducing the desired tonal character of the original sound.

In order to create an audible signal from the digital representation of the sound according to this embodiment, a digital to analog converter is used to convert the digital representation of the sound to two synchronous series of voltages, one series for frequency and the other series for amplitude. An audio generator is used to create a variable frequency sinusoidal output from the voltages in the frequency series. A variable attenuator is used to vary the amplitude of the sinusoid from the voltages in the amplitude series. The resulting signal is then amplified and audibly emitted through an audio transducer or speaker. By adjusting the amplification of the signal, the volume of the audio output may be changed as desired. For example, in some instances a low volume may be desirable to avoid frightening away birds of interest; in other situations, a high volume may be desirable to attract birds, such as with bird mating calls.

In another embodiment, the digitized image is processed as follows. Sonograms representing bird-songs will typically include frequencies from approximately 500 Hertz up to about 8,000 Hertz over a duration or interval of 2.5 seconds. However, the present invention is preferably designed to process a range of frequencies greater than that, for example, from 100 Hertz to 12,000 Hertz. The digitized image is comprised of a series of side-by-side columns of pixels, each pixel having an amplitude value. Each pixel in a vertical column is sequentially assigned to a discrete frequency starting at 100 Hertz and finishing at 12,000 Hertz in steps of 100 Hertz going up the column. In a read only memory element, or ROM, is a numerical representation of a sine-wave in list form. In a random access memory element, or RAM, is created a numerical representation of one complete cycle of a complex waveform for a particular column. Starting from the bottom of a column, the amplitude value for the first pixel in the column, at a vertical position corresponding to a frequency of 100 Hz, is multiplied by each entry in the sine-wave list and each product is transferred sequentially into the RAM as the first part of a complex waveform. The amplitude value of the next pixel in the column, at a vertical position corresponding to a frequency of 200 Hz, is multiplied by each second sine-wave entry and each product is added to the corresponding entry of the complex waveform. The amplitude value of the third pixel in the column, at a vertical position corresponding to a frequency of 300 Hz, is multiplied by each third sine-wave entry and each product is added to the corresponding entry of the complex waveform. This process progressively repeats for each pixel in the column. At this point, the construction of the complex waveform for the column is complete. An audio signal may then be output for the column and the process can then be repeated for each remaining column in the series. Alternatively, the complex waveforms for each column can be assembled into a digital representation of the sound for storage and later playback.

To generate an audible signal from the digital representation of the sound according to this embodiment, the complex waveform entries for each portion of the digital representation of the sound corresponding to an image column are transferred to a digital-to-analog converter at a rate that causes the entire complex waveform for the column to appear at the output of the digital-to-analog converter at a repetition rate of 100 Hz or once every 0.01 second. This process is completed sequentially for each portion of the digital representation of the sound corresponding to an image column to thereby reproduce the entire sound.

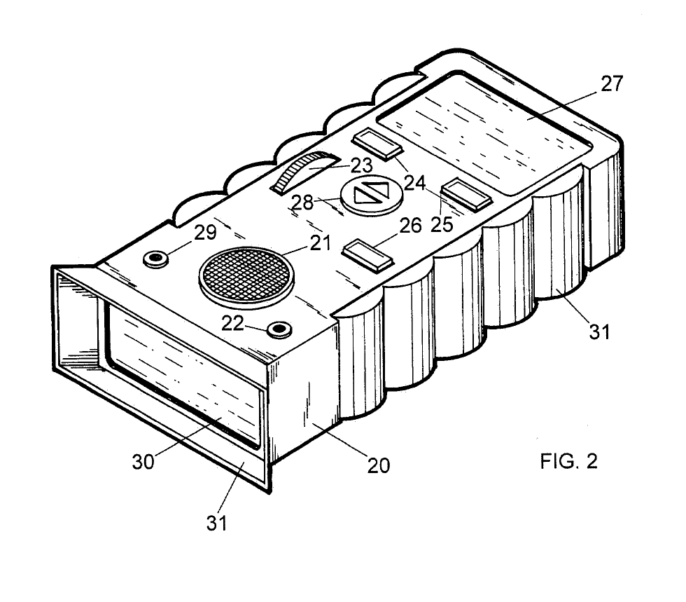

An embodiment of an apparatus according to the present invention may be described with reference to FIG. 2. A housing 20, sized to be held in one hand, is provided. The housing may be water resistant and may incorporate ergonomic features, such as comfort hand grips 31. The housing 20 has an audio output transducer 21 mounted therein. An audio output jack 22 is also provided for receiving a suitable pronged adapter to be used for connection of an external earpiece (not shown) to the housing. Typically, when an earpiece is connected to audio output jack 22, the audio output transducer 21 is disabled in favour of the audio output transducer in the earpiece. A volume wheel 23 is provided to control the amplifier portion of the audible signal generator and regulate the volume of sound produced by the audio output transducer 21. A plurality of buttons is provided on the housing. An image acquisition button 24 is provided to begin the image acquisition process. An audible output button 25 is provided to cause a digital representation of the sound to be converted to an audible output. A store button 26 is provided to store a digital representation of the sound for later playback. A screen 27 is provided in order to display a plurality of stored digital representations of sounds and a scroll button 28 is provided to allow a user to select a particular digital representation of a sound from the screen for audible output when the audible output button 25 is depressed. A data output jack 29 is provided for receiving a suitable pronged adapter to be used for connecting the housing to a data receiving device (not shown), for example, a PDA, a computer, or an Internet connection.

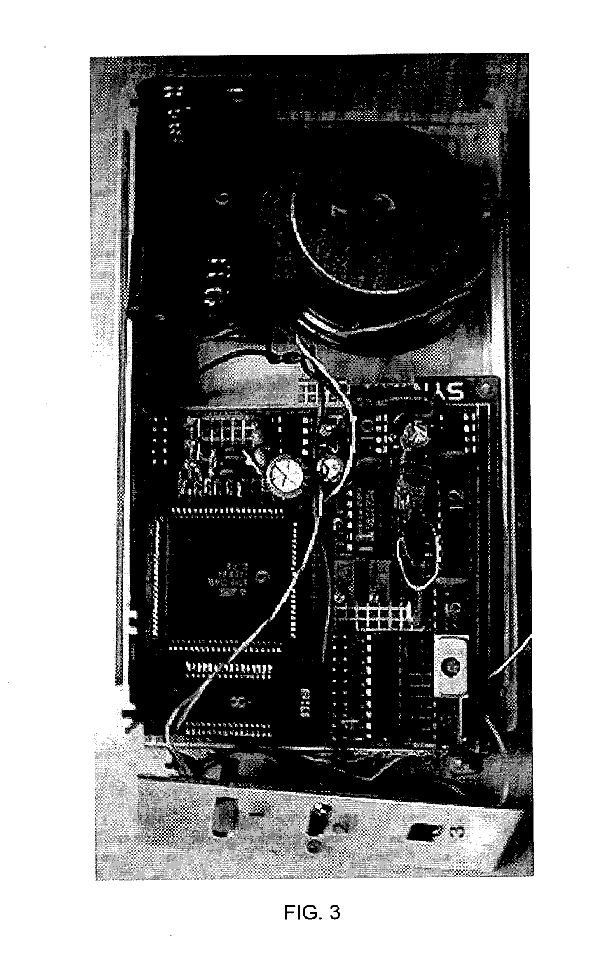

FIG. 3 shows the interior of an embodiment of an apparatus according to the present invention. The device includes operator controls, such as a volume control wheel 1, ON/OFF switch 2, and start button 3. An electrical power source 6 provides power to the device. An optical image acquisition element (not shown) may be contained within a separate but associated housing (not shown) and connected using the camera connector 4. Optically acquired digitized images are temporarily stored within a Random Access Memory (RAM) memory buffer 8. A microprocessor 5 is programmed to process the stored digitized image, converting it into a digital representation of the sound as generally described above. The digital representation of the sound is stored in a storage location. The storage location may be on the memory element 8, but could also be on another memory element (not shown) that is either inside or outside of the housing. A programmable logic device 9 performs supporting functions. The audio output amplifier 10 is used to amplify the signal prior to being emitted by the audio output transducer 7. A voltage controlled oscillator 11 and digital to analog converter 12 are used to process the numerical data into an audio signal as previously described.

With reference again to FIG. 2, an optical image acquisition element (not shown) is mounted behind lens 30 within the housing 20. A shroud 31 extends downwardly and outwardly from the bottom of the housing. The shroud 31 may be positioned over the printed sonogram or over a portion of the printed sonogram to block out external light sources. The shroud may be illuminated by a light source (not shown) that may be located within the housing behind the lens 30. The shroud 31 may also be used as a means of aligning the apparatus with the sonogram. The shroud 31 may be fixed with reference to the housing 20 or may have a telescoping movement with reference to the housing in a manner that will be more thoroughly described hereinafter.

In one embodiment, the optical image acquisition element is a monochrome video camera with a 384×288 CMOS sensor and a digital parallel port interface. The camera may be fitted with a lens capable of focussing onto the sensor a rectilinear image of a flat surface, such as a page or part of a page that has a sonogram printed on it that is placed before the lens. Single word values representing intensity of pixels appear sequentially at fixed time intervals on the parallel port of the camera in a left to right, top to bottom raster and are output to a memory buffer.

In another embodiment, the optical image acquisition element comprises a linear array of photodetectors, preferably a contact image sensor, and is positioned within a housing placed in contact with or immediately above the printed sonogram. The housing preferably incorporates a straight edge bearing a centrally located “fiducial” mark on a visible outer portion to aid in proper alignment and tracking while processing an acquired digitized image of a sonogram. A light source or array of light sources is arranged to illuminate a portion of the printed sonogram under the image sensor with uniformly diffuse light of constant and sufficient brightness to allow the contact image sensor to adequately respond to the range of intensities of the strokes on the sonogram. In use, the housing is held in one hand and positioned over a portion of the printed sonogram with an edge of the housing immediately adjacent to, and parallel with, the left edge of the sonogram. The housing is then moved to the right along the ordinate axis of the sonogram while also maintaining the position of the “fiducial” mark over the horizontal center-line of the sonogram.

While positioned over the printed sonogram, the array of photodetectors is responsive to the light reflected by the portion of the sonogram that is directly under the array. Each individual photodetector develops a voltage proportional to the intensity of light reflected by the portion of the printed sonogram directly under it. The voltages developed by all the photodetectors are assembled as an aggregate and transmitted sequentially to an analog-to-digital converter, the output of which is a digitized image that may be immediately processed as each portion is transmitted or may be temporarily stored in a memory buffer for subsequent analysis.

Sonograms in guidebooks tend to have an elongated horizontal or time axis, as compared to the vertical or frequency axis. As used in the present invention, standard optical image acquisition elements have the reverse relationship between axes; the vertical axis is longer than the horizontal axis. Although the optical image acquisition element can be oriented such that the elongate axis is horizontal, an inequality normally still exists between the aspect ratio of the axes of the sonogram and the optical image acquisition element. In order to compensate and to maximize the number of “useful” pixels in each column, there is a need to optically expand one of the axes of the sonogram such that the resolved image matches the aspect ratio of the optical image acquisition element. For example, when the optical image acquisition element is a video input device having 288 pixels in width and 384 pixels in height is turned 90 degrees, the width to height aspect ratio is 4:3; a guidebook sonogram typically has a width to height aspect ratio of 3:1, thereby requiring an expansion of 2.25 times in the height axis to make the aspect ratios match.

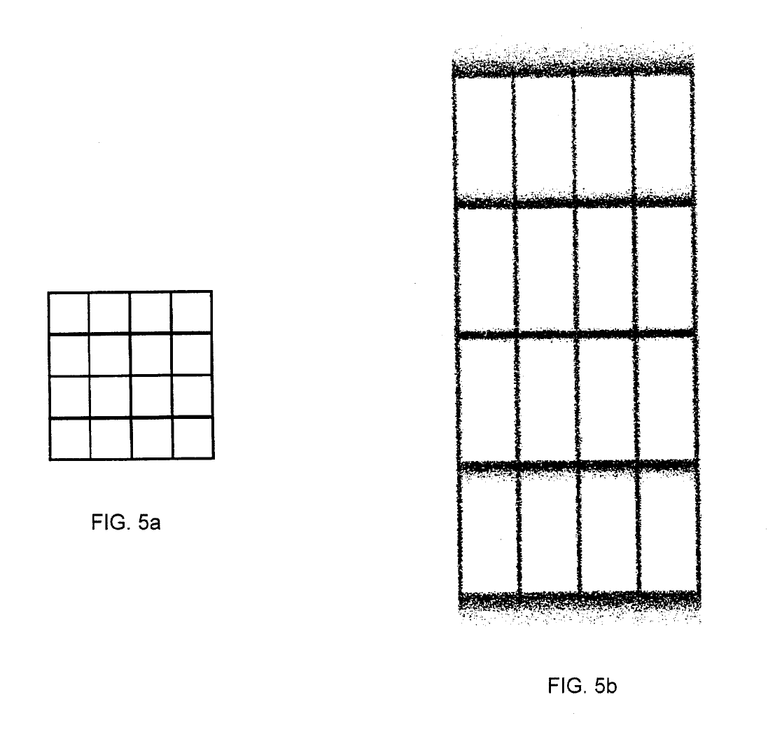

The expansion of the vertical axis is further illustrated with reference to FIGS. 5a & 5b. A square grid object is shown in FIG. 5a. The object shown in FIG. 5a is vertically expanded by two times using a Bravais anamorphic lens as previously described. FIG. 5b shows the resulting expanded image. A Bravais anamorphic lens of this type may be used to expand the vertical axis of the sonogram in order to match its aspect ratio with that of the optical image acquisition element.

The position of the lens is an important factor in obtaining a high quality image. A lens should be selected with a depth of field greater than or equal to the distance between the lens and the page in order that the entire expanded image incident to the optical image acquisition element is within focus. Usually, the optical image acquisition element is equipped with its own flat field lens and the distance between the anamorphic lens and the flat field lens is selected so as not to distort the incident image. The anamorphic lens properties and the distance between the lens and the sonogram determines the amount of vertical expansion and must be selected to match the aspect ratios; for example, a standard guidebook sonogram with an aspect ratio of 3:1 would require a certain lens position from the page, typically 8-15 cm, preferably 10 cm, to match the 4:3 aspect ratio of the optical image acquisition element.

For a standard guidebook sonogram, the desired distance between the optical image acquisition element and the lens may be pre-determined and both the lens and the optical image acquisition element may be fixedly mounted within a housing. A shroud that projects downwardly from the housing may be rested against the printed sonogram for positioning the lens at a pre-determined distance from the printed sonogram, thereby ensuring both adequate magnification and focus of the image on the optical image acquisition element.

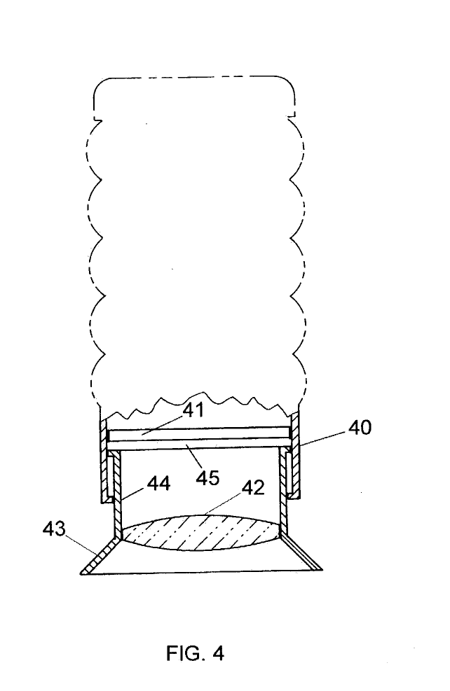

Optionally, as shown in FIG. 4, a housing 40 may include means for varying the distance between the optical image acquisition element 41 and the lens 42 to permit a user to focus the resulting image. A shroud 43 is attached to the bottom of the housing 40 and has an upwardly extending portion 44 that may telescope within the housing. The lens 42 is mounted within the shroud 43. By telescoping the shroud 43 within the housing 40, the distance between the optical image acquisition element 41 and the lens 42 may be varied. If the lens 42 is an anamorphic lens, a second lens 45 may be provided as part of the optical image acquisition element 41 to aid in resolving the image. The downward end of the shroud 43 may include a guide (not shown), so that when the left and bottom edges of the sonogram are aligned against two perpendicular edges of the guide, fixed references are provided to locate the x- and y-axes of the sonogram.

Although described with reference to the audible reproduction of bird calls in the field of ornithology, the present invention may be used to audibly reproduce any printed sonogram and is accordingly not limited by the aforementioned example of its application.

From the foregoing, it will be seen that this invention is one well adapted to attain all the ends and objects hereinabove set forth together with other advantages which are obvious and which are inherent to the structure.

It will be understood that certain features and sub-combinations are of utility and may be employed without reference to other features and sub-combinations. This is contemplated by and is within the scope of the claims.

Since many possible embodiments may be made of the invention without departing from the scope thereof, it is to be understood that all matter herein set forth or shown in the accompanying drawings is to be interpreted as illustrative and not in a limiting sense.

1. A method comprising:
a) providing a printed sonogram corresponding to a sound, the printed sonogram comprising visible strokes corresponding to the frequency and amplitude of sounds occurring at a given time;
b) optically acquiring a digitized image of the sonogram; and,
c) processing the digitized image to convert the digitized image to a digital representation of the sound.

2. The method according to claim 1, wherein the method further comprises storing the digitized image in a memory buffer.

3. The method according to claim 1, wherein the digitized image comprises a series of side by side columns, each column having a plurality of vertically stacked pixels, each pixel having an intensity, and wherein the digitized image is processed to determine frequency, amplitude and time data for each stroke based on the intensity of the pixels.

4. The method according to claim 4, wherein the digitized image is processed by:
a) determining a numerical intensity value for each pixel in a first column of the series;
b) determining the presence of a columnar section of a stroke on the digitized image based on the numerical intensity value of the pixels in the column and recording time data corresponding to the lateral position of the column on the digitized image;
c) determining the midpoint of the columnar section of the stroke and recording frequency data corresponding to the height of the midpoint on the digitized image;
d) summing the numerical intensity values of each pixel in the columnar section of the stroke and recording amplitude data corresponding to the summed numerical intensity values;
e) forming a numerical list of data for the column containing the recorded frequency, time and amplitude data; and,
f) repeating steps a) to e) for each of the columns in the series and assembling the numerical list of data for each column into a digital representation of the sound.

5. The method according to claim 1, wherein the digital representation of the sound is formatted for use with a personal computer.

6. The method according to claim 1, wherein the method further comprises storing a plurality of digital representations of sounds in a storage location.

7. The method according to claim 1, wherein the method further comprises generating an audible signal from the digital representation of the sound to thereby audibly reproduce the sound.

8. The method according to claim 7, wherein the method further comprises storing a plurality of digital representations of sounds in a storage location and wherein the audible signal is generated from a digital representation of a sound selected from the storage location.

9. An apparatus comprising:
a) a housing, the housing sized to fit in one hand;
b) an electrical power source mounted within the housing for providing electrical power to the apparatus;
c) an optical image acquisition element for optically acquiring a digitized image of a printed sonogram corresponding to a sound desired to be reproduced, the printed sonogram comprising visible strokes corresponding to the frequency and amplitude of sounds occurring at a given time; and,
d) a microprocessor mounted within the housing, the microprocessor for processing the digitized image and programmed to convert the digitized image to a digital representation of the sound.

10. The apparatus according to claim 9, wherein the apparatus further comprises a memory element mounted within the housing, the memory element comprising a memory buffer for storing the digitized image.

11. The apparatus according to claim 9, wherein the digitized image comprises a series of side by side columns, each column having a plurality of vertically stacked pixels, each pixel having an intensity and wherein the microprocessor is programmed to determine frequency, amplitude and time data for each stroke based on the intensity of the pixels.

12. The apparatus according to claim 11, wherein the microprocessor is programmed to:
a) determine a numerical intensity value for each pixel in a first column of the series;
b) determine the presence of a columnar section of a stroke on the digitized image based on the numerical intensity value of the pixels in the column and record time data corresponding to the lateral position of the column on the digitized image;
c) determine the midpoint of the columnar section of the stroke and record frequency data corresponding to the height of the midpoint on the digitized image;
d) sum the numerical intensity values of each pixel in the columnar section of the stroke and record amplitude data corresponding to the summed numerical intensity values;
e) form a numerical list of data for the column containing the recorded frequency, time and amplitude data; and,
f) repeat steps a) to e) for each of the columns in the series and assemble the numerical list of data for each column into a digital representation of the sound.

13. The apparatus according to claim 9, wherein the apparatus further comprises an audible signal generator for generating an audible signal from the digital representation of the sound to thereby reproduce the sound.

14. The apparatus of claim 13, wherein the audible signal generator comprises a digital to analog converter, an amplifier, and an audio output transducer.

15. The apparatus according to claim 14, wherein the audio output transducer is mounted within an ear piece.

16. The apparatus according to claim 9, wherein the optical image acquisition element comprises a video input device, a CCD, a photodetector array, or a line scanner.

17. The apparatus according to claim 16, wherein the optical image acquisition element is mounted within the housing.

18. The apparatus according to claim 9, wherein the optical image acquisition element comprises a downwardly oriented uniform array of photodetectors positioned above a portion of the printed sonogram and a lens positioned between the array and the portion of the printed sonogram, the lens for focussing an optical image of the printed sonogram onto the array.

19. The apparatus according to claim 18, wherein the distance between the lens and the array is adjustable.

20. A method comprising:
a) making a preliminary identification of a bird based on hearing a first sound produced by the bird;
b) selecting a printed sonogram based on the preliminary identification of the bird from a book having a plurality of sonograms cross referenced with bird identities, the printed sonogram comprising visible strokes corresponding to the frequency and amplitude of sounds occurring at a given time, the printed sonogram corresponding to a second sound;
c) optically acquiring a digitized image of the sonogram;
d) storing the digitized image in a memory buffer;
e) processing the digitized image to convert the digitized image to a digital representation of the second sound;
f) generating an audible signal from the digital representation of the second sound to thereby audibly reproduce the second sound; and,
g) comparing the second sound with the first sound to confirm the preliminary identification of the bird.
